(19) United States
(12) Patent Application Publication
(10) Pub. No.: US 2013/0338729 A1
(43) Pub. Date: Dec. 19, 2013

(54) SELF-CONTAINED ADHESIVE PATCH FOR ELECTRICAL STIMULATION FOR PAIN RELIEF AND MUSCLE FATIGUE

(71) Applicant: Donald SPECTOR, New York, NY (US)

(72) Inventor: Donald SPECTOR, New York, NY (US)

(21) Appl. No.: 13/571,705

(22) Filed: Aug. 10, 2012

Related U.S. Application Data
(60) Provisional application No. 61/660,717, filed on Jun. 16, 2012.
(60) Provisional application No. 61/660,779, filed on Jun. 17, 2012.

(51) Int. Cl. A61N 1/36

(52) U.S. Cl. 607/46

(57) ABSTRACT

An electrical stimulation device has a flexible base, an adhesive applied to a bottom side of the base and an electrical stimulation device connected to the base. The electrical stimulation device comprises a power source, an electrical stimulator connected to the power source, and a controller for turning the power source on and off.

### CROSS-REFERENCE TO RELATED APPLICATIONS

This application claims priority under 35 U.S.C. 119(e) of U.S. Provisional Applications Ser. Nos. 61/660,717 filed on Jun. 16, 2012, and 61/660,779 filed on Jun. 17, 2012.

### BACKGROUND OF THE INVENTION

1. Field of the Invention

This invention relates to a wireless electrical stimulation patch to be worn by an individual to promote pain relief and ease muscle fatigue.

2. The Prior Art

It is known that electrical stimulation can provide pain relief to sore muscles. Electrical impulses are supplied to the skin of a patient through electrodes. These impulses relieve pain and fatigue in the muscles receiving the stimulation.

Traditionally, administering electrical therapy required cumbersome machines, and wires connected to the patient. Thus the patient would typically be required to go to a therapy center to be connected to the electrical stimulation device. This can be expensive and inconvenient.

US Patent Publication No. 2009/0182393 to Bachinski tries to overcome this by providing a patch that is applied to the patient. In this invention, the patch must be connected to a controller which is then removed for recharging in a docking station. However, this patch is bulky and cumbersome, and can't be used during athletic activity.

### SUMMARY OF THE INVENTION

It is therefore an object of the invention to provide a portable flexible electrical stimulation device that can be used before, during and after athletic activity.

This and other objects are accomplished by an electrical stimulation device that has a flexible base, an adhesive applied to a bottom side of said base and an electrical stimulation device connected to the base. The electrical stimulation device comprises a power source, an electrical stimulator connected to the power source, and a controller for turning the power source on and off.

In a preferred embodiment, the power source is a battery. Any suitable small, low profile battery can be used. The batter can be rechargeable and removable, or disposable and permanently connected to the base.

The base is preferably made of woven or nonwoven fabric or plastic. It is important that the base be flexible enough to bend along the contours of a person's body when the patch is applied to the body, and to flex along with movements of the person so that the patch remains on the body during exercise.

The electrical stimulator preferably comprises electrical lead wires connected to the battery and a quantity of conductive paste attached to the flexible base. The lead wires are in contact with the conductive paste to transmit electricity from the wires through the conductive paste. The conductive paste preferably comprises silicone or epoxy resin in combination with a metal such as silver, copper, nickel and aluminum. The paste should be flexible so as to remain in contact with the person's skin at all times after the patch has been applied. The paste can also be adhesive to increase the contact possibility with the person's skin. If the paste is adhesive, it could also be used as the adhesive to apply the patch to the person. Otherwise, a separate adhesive is applied around the perimeter of the flexible base to apply the patch to a person, and the conductive paste is disposed within the perimeter.

The controller preferably comprises a wireless microprocessor connected to the battery, and a wireless control device adapted to communicate with the microprocessor to turn the battery power off and on. The wireless control device can be a smartphone or a personal computer, or can be a dedicated controller built specifically for controlling the microprocessor. If a smartphone or personal computer is used, software is installed on the smartphone or personal computer that enables the smartphone or personal computer to instruct the microprocessor to control the battery. The instructions could be as simple as on/off, or could also be used to control the level of electrical stimulation. In addition, the software could be programmed to operate the patch on a timer, so that stimulation is applied automatically at certain times and at certain levels.

The microprocessor and/or the controller can also be connected to a biometric sensor, which can be incorporated into the patch or be located elsewhere on the user. The biometric sensor could signal the controller and/or the microprocessor to turn the electric stimulation on or off based on the conditions sensed by the sensor.

### BRIEF DESCRIPTION OF THE DRAWINGS

Other objects and features of the present invention will become apparent from the following detailed description considered in connection with the accompanying drawings. It is to be understood, however, that the drawings are designed as an illustration only and not as a definition of the limits of the invention.

In the drawings, wherein similar reference characters denote similar elements throughout the several views:

FIG. 1 shows a cross-sectional view of one embodiment of the patch according to the invention with a remote control device;

FIG. 2 shows a top view of the patch with the top layer cut away; and

FIG. 3 shows the patch and control device in use on a person.

### DETAILED DESCRIPTION OF THE PREFERRED EMBODIMENT

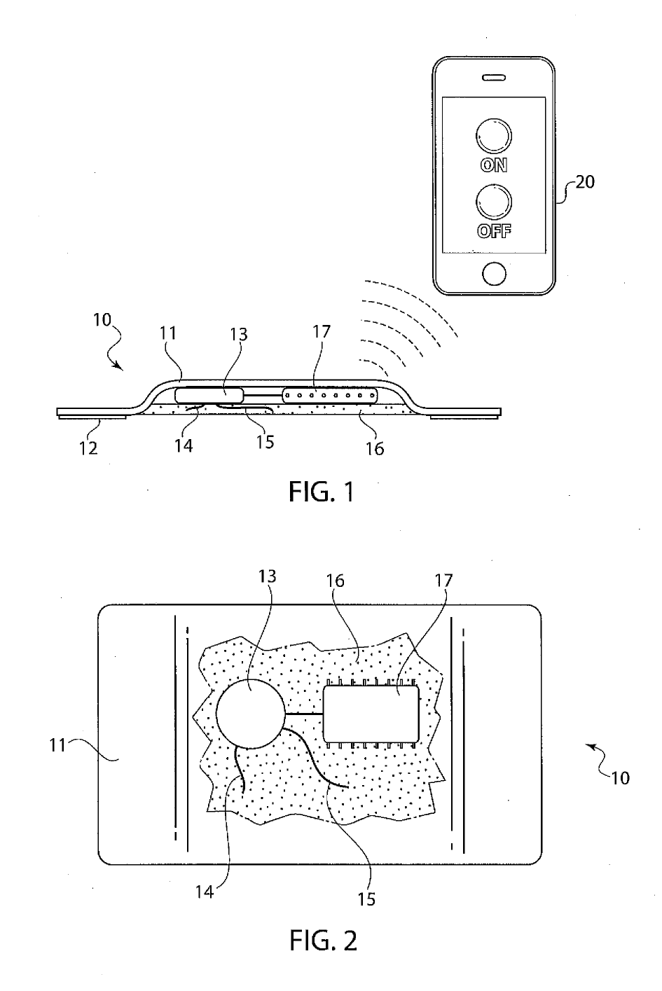

Referring now in detail to the drawings and, in particular, FIGS. 1 and 2 show an electrical stimulation patch 10 which comprises a flexible base 11 which is preferably made of woven or non-woven fabric such that the fabric can flex and/or stretch as the wearer is moving. Around the perimeter of base 11 is an adhesive layer 12 for applying patch 10 to a person. Adhesive 12 is strong enough to keep patch 10 in place, but has enough release materials present to allow patch 10 to be removed without pain or injury to the skin of the wearer.

Underneath base 11 is disposed a battery 13 with leads 14, 15 which extend into a layer of conductive paste 16. Conductive paste conducts the electricity from leads 14, 15 through to the skin of the wearer, to supply electrical stimulation to the wearer of the patch. Conductive paste 16 is preferably formed from silicone or epoxy resin, with metallic substances incorporated therein for conductivity. In particular, copper, silver, aluminum or nickel can be used. Conductive paste 16 is preferably flexible so that it remains in contact with the wearer's skin at all times during activity. Conductive paste 16 can also have adhesive properties to increase the contact between paste 16 and the wearer's skin. Adhesive layer 12 could also be formed from conductive paste 16.

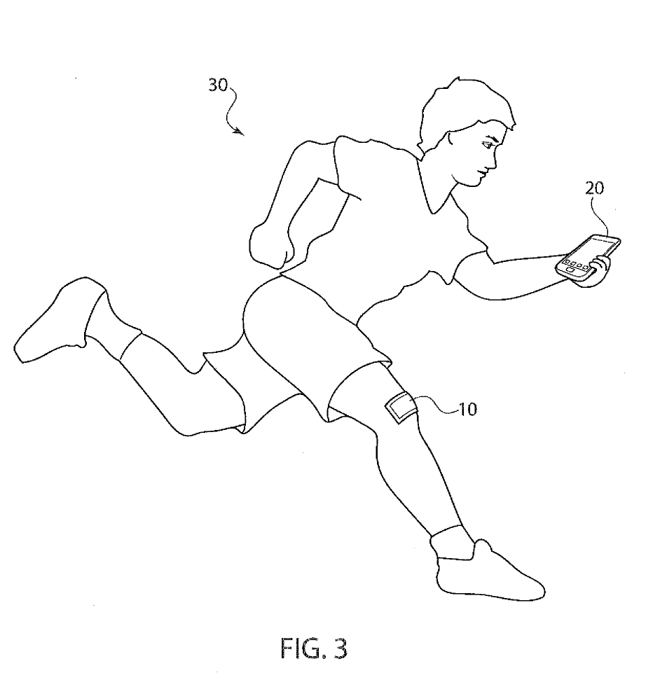

Battery 13 can be any suitable small, low profile battery. Battery 13 can be rechargeable or disposable. Connected to battery 13 is a microprocessor 17 for controlling the power of battery 13. Microprocessor 17 is preferably a wireless microprocessor that communicates with an external control system for controlling the power of battery 13. In the embodiment shown, the external control system is a smartphone 20. Smartphone 20 is loaded with software that allows smartphone 20 to instruct microprocessor 17 to turn the power of battery 13 off or on and to adjust the amount of current flowing through the leads. This allows the wearer 30 (shown in FIG. 3) to customize the duration and amount of electrical stimulation applied to the body without removing patch 10.

The patch according to the invention provides targeted electrical stimulation to any part of the body that requires it. The patch can be made in various sizes and shapes to accommodate different body parts. The battery can be made to be removable and replaceable, or the entire patch can be disposable. The amount of amount of electrical stimulation can be varied, either by the controller or by the size of the batter and leads, and the conductivity of the paste.

Accordingly, while only a few embodiments of the present invention have been shown and described, it is obvious that many changes and modifications may be made thereunto without departing from the spirit and scope of the invention.

What is claimed is:

1. An electrical stimulation device comprising:
a flexible base having a top side and a bottom side;
an adhesive applied to at least a portion of the bottom side of said base; and
an electrical stimulation device connected to the base; the electrical stimulation device comprising:
(a) a power source;
(b) an electrical stimulator connected to the power source; and
(c) a controller controlling the power source;
wherein when the based is attached to a person's skin with the adhesive, the electrical stimulator delivers electrical stimulation to the person.

2. The device according to claim 1, wherein the power source is a battery.

3. The device according to claim 1, wherein the base is made of woven or nonwoven fabric or plastic.

4. The device according to claim 1, wherein the electrical stimulator comprises electrical lead wires connected to the battery and a quantity of conductive paste attached to the flexible base, wherein the lead wires are in contact with the conductive paste to transmit electricity from the wires through the conductive paste.

5. The device according to claim 1, wherein the controller comprises a microprocessor connected to the battery, and a wireless control device adapted to communicate with the microprocessor to control the power supply from the battery.

6. The device according to claim 5, wherein the wireless control device is a smartphone or a personal computer.

7. The device according to claim 1, wherein the flexible base is made of woven or nonwoven fabric.

8. The device according to claim 4, wherein the adhesive extends around a perimeter of the base and surrounds the conductive paste.

9. The device according to claim 1, wherein the electrically conductive paste comprises silicone or epoxy resin in combination with a metal selected from the group consisting of silver, copper, nickel and aluminum.

10. The patch according to claim 5, wherein the wireless control device is adapted to turn the power from the battery off and on, and control the amount of current through the leads.
